(19) United States
(12) Patent Application Publication
(10) Pub. No.: US 2020/0005211 A1
(43) Pub. Date: Jan. 2, 2020

(54) INFORMATION PROCESSING SYSTEM

(71) Applicant: HITACHI, LTD., Tokyo (JP)

(72) Inventors: Satomi TSUJI, Tokyo (JP); Miki HAYAKAWA, Tokyo (JP); Yuuichi OGAWA, Tokyo (JP); Yuichiro TANAKA, Tokyo (JP)

(21) Appl. No.: 16/493,435

(22) PCT Filed: Sep. 11, 2017
(86) PCT No.: PCT/JP2017/032628

§ 371 Date: Sep. 12, 2019

(51) Int. Cl. G06Q 10/06; A61B 5/11

(52) U.S. Cl. CPC G06Q 10/06393; A61B 2562/0219; A61B 5/1118

(57) ABSTRACT

Provided is an information processing system which is suitable for evaluating a state of a group based on measured data of a plurality of persons. The present invention is an information processing system comprising a recording device which collects, and stores, data via a network from a terminal device worn by each of a plurality of persons, and a computer which sets a group of a predetermined number of persons among the plurality of persons based on the collected data, wherein the terminal device outputs measured data of the person to the network, wherein the recording device stores the measured data, and wherein the computer calculates an index of a state of activity of the group based on the measured data of the terminal device worn by each person belonging to the group.

### TECHNICAL FIELD

The present invention generally relates to an information processing system, and particularly relates to a system of using a computer for evaluating the characteristics of a group, such as a working group, based on measured data.

### BACKGROUND ART

In recent years, big data is attracting attention, and activities of analyzing the data acquired in a business system, discovering, based on quantitative statistical analysis, factors which will affect the indexes (such as profits, manufacturing time and manufacturing cost) to become the company's KPI, and utilizing the results in the decision-making of corporate activities are being conducted.

It is known that a person's state of mind (for instance, stress or flow state) affects that person's productivity, and, for example, NPTL 1 describes that, when people are separated into a group of persons with a healthy state of mind and a group of persons in a depressed state, there will be a difference in productivity between the two groups.

Furthermore, PTL 1 discloses a system of causing an individual to wear a wearable sensor node comprising acceleration sensors in a triaxial direction, and calculating the acceleration rhythm based on the observed data. This system determines whether the individual is in a state of activity or a state of rest, additionally obtains the distribution profile, specifically the inclination and inflection point, in the histogram of the duration of the state of activity, and thereby estimates the stress of the individual.

Furthermore, similar to PTL 1, PTL 2 also discloses a technology of easily estimating the stress of an individual based on a linear sum of the incidence ratio of a specific scope in the distribution profile of the duration of that individual's state of activity.

### CITATION LIST

### Patent Literature

[PTL 1] Japanese Patent No. 5588563
[PTL 2] International Publication No. 2016/125260

### Non-Patent Literature

[NPTL 1] Nakamura, Toru et al., “Universal Scaling Law in Human Behavioral Organization”, Physical review letters, pp. 138103-1-4, 2007

### SUMMARY OF THE INVENTION

### Problems to be Solved by the Invention

The superiority or inferiority of a person's productivity is not dependent only on the responsibilities of that person, and is also significantly affected by the ambient environment. For example, it is known that a person's productivity will change depending on the voice or mood of the person in the same space or the state of mind of the conversation partner. More specifically, in a meeting for coming up with new ideas, a tolerant atmosphere where more opinions are given would result in more ideas in comparison to a quite and critical atmosphere. Meanwhile, in accounting work in which accuracy is required, it is easy to imagine that a more quiet and tense atmosphere would be more preferable.

Nevertheless, with the conventional systems, while it is possible to evaluate and determine the state of activity of an individual based on sensors which measure the state of that individual, no consideration was given to evaluating the superiority or inferiority of the state of a group consisting of a plurality of persons.

If it is possible to index the state of a group, such as whether a group of a plurality of persons is in a state of activation or invigoration, or a state of stagnation or calmness, it would be useful in evaluations related to the improvement in the productivity of a group such as an organization. Thus, an object of the present invention is to provide an information processing system which is suitable for evaluating a state of a group based on measured data of a plurality of persons.

### Means to Solve the Problems

In order to achieve the foregoing object, the present invention is an information processing system comprising a recording device which collects, and stores, data via a network from a terminal device worn by each of a plurality of persons, and a computer which sets a group of a predetermined number of persons among the plurality of persons based on the collected data, wherein the terminal device outputs measured data of the person to the network, wherein the recording device stores the measured data, and wherein the computer calculates an index of a state of activity of the group based on the measured data of the terminal device worn by each person belonging to the group. The present invention additionally provides a method for realizing this information processing system.

### Advantageous Effects Of The Invention

According to the present invention, it is possible to provide an information processing system which is suitable for evaluating a state of a group based on measured data of a plurality of persons.

### BRIEF DESCRIPTION OF DRAWINGS

FIG. 1 is a block diagram showing an embodiment of the information processing system.

FIG. 2 is a block diagram showing an embodiment of the terminal.

FIG. 3 is a block diagram showing an embodiment of the configuration of the sensor net server and the base station.

FIG. 4 is a table showing an embodiment of the sensing database (acceleration data).

FIG. 5A is a table showing an embodiment of the sensing database (face-to-face data).

FIG. 5B is a table showing another embodiment of the sensing database (face-to-face data).

FIG. 6 is a table showing an embodiment of the acceleration frequency table.

FIG. 7 is a block diagram showing an embodiment of the configuration of the client device, the application server, and the position detection sensor.

FIG. 8 is a table showing an embodiment of the group state data (index).

FIG. 9 is a table showing an embodiment of the group state data (index).

FIG. 10 is an example of a diagram showing a user attribute list.

FIG. 11 is a table showing an embodiment of the area determination data and the proximity determination data.

FIG. 12 is an example of a sequence diagram showing the calculation routine in the application server.

FIG. 13 is an example of a flowchart for calculating the group state index.

FIG. 14 s a table showing an example of the scope of calculation.

FIG. 15 is an example of a histogram of the activity duration.

FIG. 16 is a table showing another example of the scope of calculation.

FIG. 17A is an example of a screen of the Web application displaying the group state index.

FIG. 17B is another example of a screen of the Web application displaying the group state index.

FIG. 17C is yet another example of a screen of the Web application displaying the group state index.

FIG. 18 is another example of a flowchart for calculating the group state index.

FIG. 19 is yet another example of a screen of the Web application displaying the group state index.

FIG. 20 is yet another example of a screen of the Web application displaying the group state index.

FIG. 21 is a table showing an example of the degree of involvement of the group members.

FIG. 22 is a table showing another example of the degree of involvement of the group members.

FIG. 23 is yet another example of a flowchart for calculating the group state index.

FIG. 24 is yet another example of a screen of the Web application displaying the group state index.

### DESCRIPTION OF EMBODIMENTS

The present invention is an information processing system for evaluating, determining or judging various states of a group a plurality of persons (individuals), such a state in which the group is active or invigorated, or contrarily a state in which the group is calm or highly stressed. The information processing system measures the state of persons, such as the person's movement or vitals, via a sensor, and evaluates the state of the group based on the measured value. The sensor may be a wearable sensor to be worn by the person. The term “person” may also be referred to as a solid body including humans and animals.

The information processing system finds a “practical contact” based on predetermined standards, rules or requirements from the measured data of each of the plurality of persons, and defines the group based thereon. Furthermore, the information processing system integrates the measured data of each of the plurality of persons belonging to the group, extracts the characteristics of the integrated data, and obtains an index of a state of the group. The information processing system uses the obtained index for evaluating the state of the group.

A state in which the group is active or invigorated is a state which yields an environment in which good influence is exerted not only on the individual, but also on the entire organization, for the individual to concentrate and conduct activities as a result of independent actions such as a plurality of persons gathering and engaging in lively discussion, the supervisor praising subordinates for their work, or individuals making idle conversation during their break.

A group may include a so-called fixed or regular group defined based on an office organization such as the company's business department, division or section, as well as a so-called dynamic, temporary or irregular group that transverses the office organization such as a working group or a project group.

The information processing system can define, select or determine a group, and evaluate the state of the determined group based on the measured values of the sensor. For example, the information processing system can define the plurality of persons included in the group as the calculation target and the time range of calculation based the state of proximity of people and/or information regarding the staying area of such people (hereinafter referred to as the predetermined reference), and index the state of the group by using the measured data of the terminal (device) worn by each of the plurality of persons within the foregoing range.

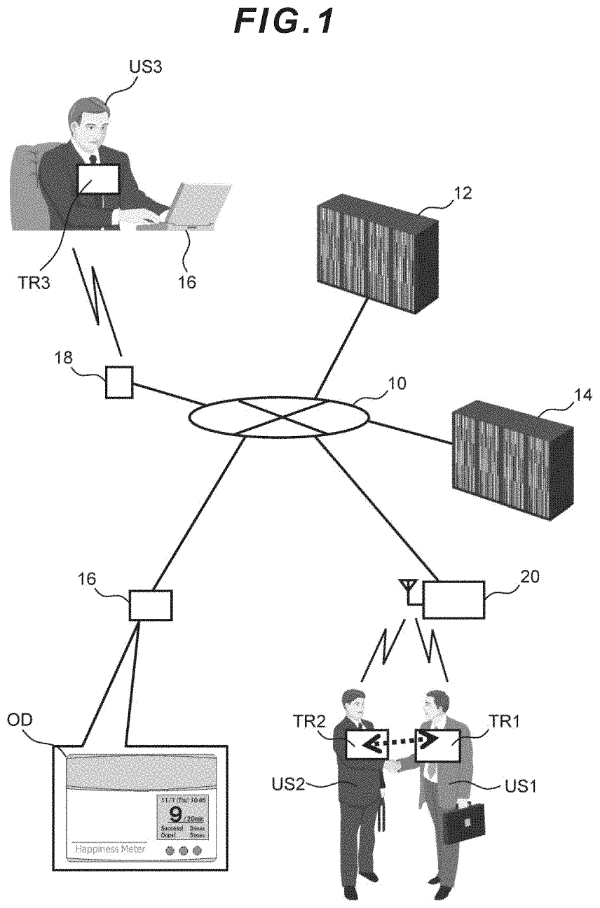

The information processing system can be broadly classified as comprising a device for collecting, and storing, information from the sensor, and a computer for analyzing, evaluating or determining the state of the group based on the information from the sensor. FIG. 1 is a block diagram showing an example of the information processing system. In the information processing system centered around a network 10, a base station 20 which incorporates sensing data from the terminal (TR: TR1 to 2) worn by each user (US: US1 to 2) staying within an area of the system, a position detection sensor 18 which detects the location of the terminal, a sensor net server (recording device) 14, and an application server (computer) 12 are connected to the network 10. Furthermore, a client terminal 16 of the administrator is connected to the network 10.

The terminal TR is worn by the user. The terminal acquires data related to body movement and data related to the face-to-face state (interaction) with other wearers. The means for the former may be, for example, an acceleration sensor. The acceleration sensor provides triaxial acceleration data related to body movement to the microcomputer within the terminal. The means for the latter may be, for example, an infrared transmission/reception circuit. When the users approach each other or face each other, infrared rays are transmitted/received between the respective terminals. The means for the latter may also be realized with a close-range wireless transmission/reception device, or the terminal's camera and a facial recognition program.

Because the terminal TR and the base station 20 are connected wirelessly, each of a plurality of terminals TR connects to a nearby base station and forms a personal area network (PAN). As a result of the infrared transmission/reception circuit transmitting/receiving infrared rays between the terminals, it is detected whether a terminal is facing another terminal; that is, whether a person wearing the terminal is facing another person wearing a different terminal. Thus, the terminal is desirably worn on the front side of the person.

The position detection sensor 18 provides a means for determining whether a terminal (TR3) of a user (US3) is nearby, or determining the staying area of the terminal or whether the terminal is staying in a specific area. This means may be the same as the foregoing “means for the latter”.

The terminal is connected to the base station 20 and the position detection sensor 18 based on wireless or wired connection. The base station 20 transmits the data, which was transmitted from the terminal, to the sensor net server 14 via the network 10. The sensor net server 14 accumulates and stores data. The same applies to the position detection sensor 18.

The application server 12 periodically acquires data from the sensor net server 14, and calculates an index related to the stage of the group in predetermined time units. The group may be a gathering of a plurality of individuals linked based on predetermined rules, a predetermined relationship, or a predetermined purpose. A state of the group may be a group attribute such as whether the group has vigor or whether the group exhibits cooperativeness. An index may be a value or a parameter which represents the evaluation. The client terminal 16 displays, on a screen (OD), the index of the group state acquired from the application server 12. The result of performing correlation analysis, through association with other business data as needed, may also be displayed on the screen. The application server 12 and the sensor net server 14 are examples of a computer system.

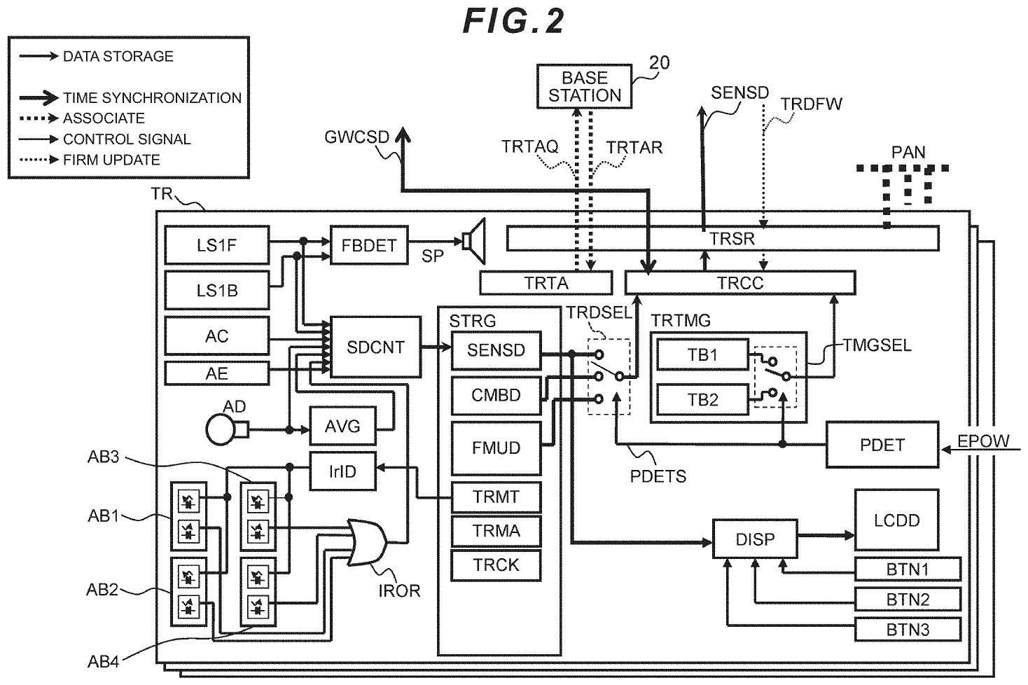

The detailed configuration of the constituent elements of the system is now explained. FIG. 2 is a block diagram showing an example of the terminal as a sensor node. The terminal can be broadly classified as comprising a control module, a storage module, and a transmission/reception module. The control module is configured from a CPU as the control resource of the computer, and the storage module is configured from a storage resource such as a semiconductor storage device or a magnetic storage device. The transmission/reception module is configured from a wired or wireless network interface. Otherwise, the terminal may also comprise a peripheral device such as a timekeeper.

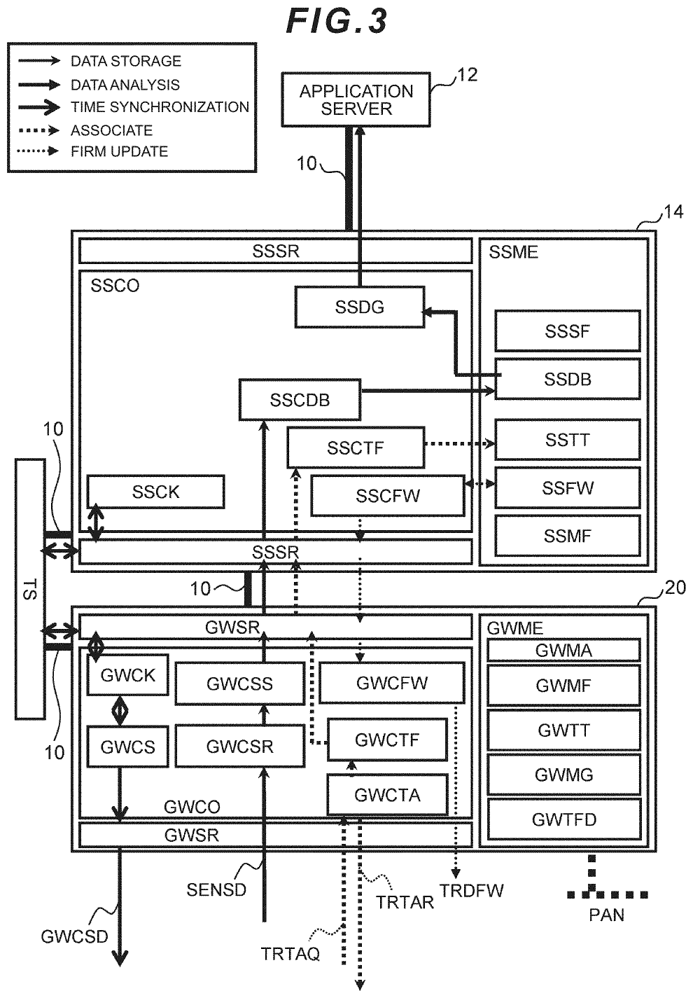

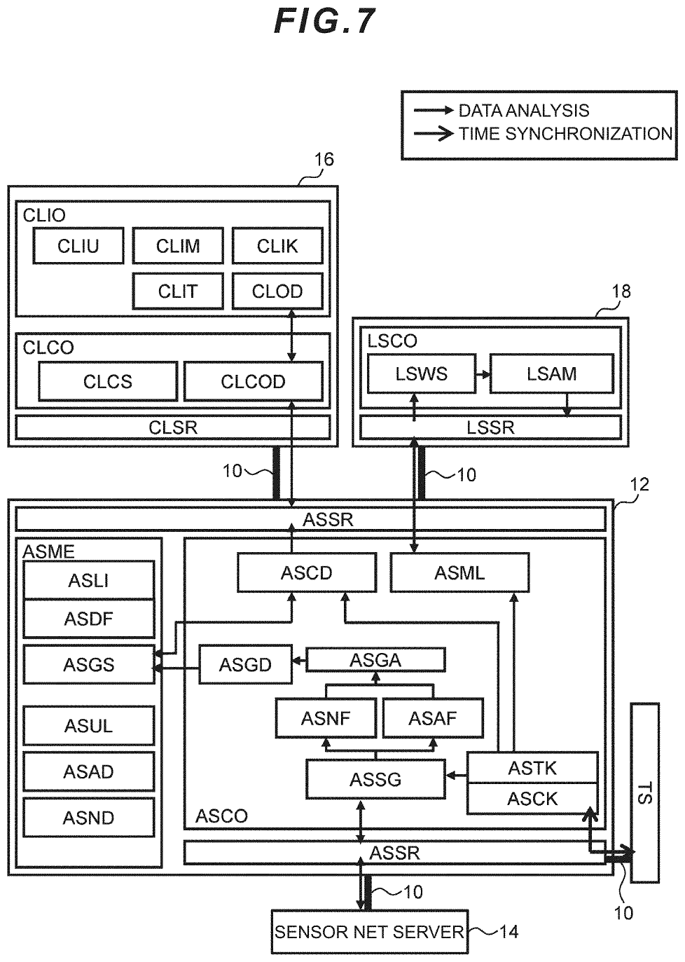

The respective blocks shown in FIG. 2 show a module realized with hardware, a module realized with software, or a module realized through the coordination of hardware and software. The 6 different types of arrows in FIG. 2 respectively represent time synchronization, association, storage of acquired sensing data, analysis of sensing data, firmware update, and data for control signals or flow of signals. These are also the same in FIG. 3 and FIG. 7 described later.

The terminal may also be, for example, of a card type so that it can be easily worn or carried by the individual. The terminal comprises a plurality of infrared transmission/reception modules (AB: AB1-4), a triaxial acceleration sensor (AC), a microphone (AD) which detects the wearer's speech and peripheral sound, and a plurality of sensors such as illuminance sensors (LS1F, LS1B) and a temperature sensor (AE) for detecting both sides of the terminal. The terminal's temperature sensor (AE) acquires the temperature of the place where the terminal is located, and the illuminance sensor (LS1F) acquires the illuminance of the surface where the terminal is facing. The terminal is thereby able to record its ambient environment. For example, it is also possible to know that the terminal moved from a certain place to another place based on the temperature and illuminance.

The terminal comprises four infrared transmission/reception modules (AB: AB1-4). The infrared transmission/reception module (AB) periodically transmits terminal information (TRMT), which is unique identifying information of the terminal, toward the front direction. When a person wearing a different terminal is positioned roughly in front (for instance, front side or obliquely front side), the terminal and the other terminal mutually transmit/receive their respective terminal information (TRMT) via infrared communication. Accordingly, the system can record who and who are facing each other based on information from the two terminals.

The terminal transmits terminal information (TRMT) and location information to the position detection sensor (18: FIG. 1) installed at a predetermined position of the user's activity environment. Accordingly, the system can detect a terminal (user) staying in a predetermined area.

The infrared transmission/reception module (AB) comprises an infrared light emitting diode, and an infrared phototransistor. The infrared ID transmission module (IrID) generates ID information (TRMT) of the terminal and forwards the generated ID information (TRMT) to the infrared light emitting diode of the infrared transmission/reception module. The same data is transmitted to a plurality of infrared transmission/reception modules, and all infrared light emitting diodes light up simultaneously. Otherwise, the same or different data may be output at an independent timing to each of the plurality of infrared transmission/reception modules.

A logical sum circuit (IROR) acquires a logical sum from the data of a plurality of infrared phototransistors. In other words, so as long as at least one infrared transmission/reception module has received the terminal ID, the terminal will recognize another terminal. Note that the terminal may also independently comprise a plurality of reception circuits in substitute for the logical sum circuit (IROR). In this mode, because the terminal can comprehend the transmission/reception state of each of the plurality of infrared transmission/reception module, for example, it is also possible to obtain additional information such as in which direction the other terminal, which is to face the terminal, is located.

A sensing data storage control module (SDCNT) stores, in a storage module (STRG), sensing data (SENSD) detected by the sensor. A communication control module (TRCC) processes the sensing data (SENSD) into a transmission packet, and the transmission/reception module (TRSR) transmits the transmission packet to the base station (GW).

Here, the communication timing control module (TRTMG) extracts the sensing data (SENSD) from the storage module (STRG), and determines the timing of the wireless or wired transmission. The communication timing control module (TRTMG) has a plurality of time bases (TB1, TB2) for determining a plurality of timings.

As the data to be stored in the storage module (STRG), in addition to the sensing data (SENSD) detected by the sensor immediately before, there are collectively transmitted data (CMBD) accumulated in the past and firmware update data (FMUD) which is an operation program of the terminal for updating the firmware.

An external power supply connection detection circuit (PDET) detects that an external power supply (EPOW) has been connected, and generates an external power supply detection signal (PDETS). A time base switching module (TMGSEL) switches the transmission timing generated by the timing control module (TRTMG) based on the external power supply detection signal (PDETS). A data switching module (TRDSEL) switches the data to be wirelessly communicated.

The time base switching module (TMGSEL) switches the transmission timing based on the external power supply detection signal (PDETS) from the two time bases of time base 1 (TB1) and time base 2 (TB2).

The data switching module (TRDSEL) switches the data to be communicated based on the external power supply detection signal (PDETS) from the sensing data (SENSD) obtained from the sensor, the collectively transmitted data (CMBD) accumulated in the past, and the firmware update data (FMUD).

The illuminance sensors (LS1F, LS1B) each exist on the front face and the back face of the terminal (TR). The sensing data storage control module (SDCNT) stores, in the storage module (STRG), the data acquired by the illuminance sensors (LS1F, LS1B), and a turnover detection module (FBDET) compares the two data. When the terminal is properly worn by a person, the illuminance sensor (LS1F) mounted on the front face receives external light, and the illuminance sensor (LS1B) mounted on the back face does not receive external light. Accordingly, the illuminance detected by the illuminance sensor (LS1F) will be of a greater value than the illuminance detected by the illuminance sensor (LS1B). Meanwhile, when the front/back of the terminal is reversed, the values will be the opposite. When the turnover detection module (FBDET) detects that the front/back of the terminal is reversed, it outputs a beep sound from a speaker (SP).

A microphone (AD) acquires sound information. The system can know the ambient environment such as “noisy” or “quiet” based on the sound information. Furthermore, as a result of acquiring and analyzing the person's voice, the system can generate a behavioral index related to face-to-face communication such as whether the communication is active or stagnant, whether the conversation is equally bidirectional or one sided, and whether the person is angry or laughing. In addition, it is also possible to complement, based on the sound information and the acceleration information, the face-to-face state which could not be detected by the infrared transmission/reception device (AB) based on the relationship of the standing position of the person.

An integrating circuit (AVG) integrates the sound waveforms acquired by the microphone (AD). The integral value corresponds to the energy of the acquired sound.

A triaxial acceleration sensor (AC) detects the acceleration of the node; that is, the movement of the node. The system can analyze the action of the person wearing the terminal, such as the intensity of the person's movement or the person's gait, from the acceleration data. Furthermore, by comparing the values of acceleration in the same time period detected by a plurality of terminals, the system analyzes the degree of activity of communication, mutual rhythm, and mutual correlation between the persons wearing those terminals.

The sensing data storage control module (SDCNT) stores, in the storage module (STRG), the data acquired by the triaxial acceleration sensor (AC).

The terminal comprises, as I/O devices, buttons 1 to 3 (BTN1 to 3), a display device (LCDD), and a speaker (SP).

The storage module (STRG) is a hard disk or a nonvolatile storage device such as a flash memory. The storage module stores the terminal information (TRMT) as the unique identifying number of the terminal, and the operation setting (TRMA) such as the sensing interval and contents to be output to the display. The storage module (STRG) can temporarily record data and, for example, records the sensed data.

A timekeeper (TRCK) retains time information (GWCSD), and updates the time information (GWCSD) in regular intervals. The timekeeper (TRCK) periodically corrects the time based on the time information (GWCSD) transmitted from the base station (20: FIG. 1) in order to prevent the time information (GWCSD) from deviating from the other terminals.

The sensing data storage control module (SDCNT) controls the sensing interval of the respective sensors and manages the acquired data according to the operation setting (TRMA) recorded in the storage module (STRG).

Time synchronization is performed by acquiring the time information from the base station (20: FIG. 1) and correcting the timekeeper (TRCK). Time synchronization may be executed immediately after the association described later, or executed according to the time synchronization command transmitted from the base station.

The communication control module (TRCC), upon transmitting/receiving data, converts the data into a data format corresponding to the control of the transmission interval and the wireless transmission/reception. The communication control module (TRCC) may comprises, as needed, a wired communication function rather than a wireless communication function. The communication control module (TRCC) may also perform congestion control so that the transmission timing does not overlap with the other terminals.

An associate (TRTA) transmits/receives an associate request (TRTAQ) for forming a personal area network (PAN) with the base station (20: FIG. 1), and an associate response (TRTAR), and thereby determines the base station to which the data should be transmitted. The associate (TRTA) is executed when the power supply of the terminal is turned on, and when the previous transmission/reception to and from the base station is disconnected due to the movement of the terminal. In the case of a wired connection, the associate (TRTA) is executed when the terminal detects that it has been connected to the base station based on a wired connection. As a result of the associate (TRTA), the terminal is associated with one base station (GW) within a close range where the wireless signal from that terminal will reach.

The transmission/reception module (TRSR) comprises an antenna, and transmits/receives wireless signals. If necessary, the transmission/reception module (TRSR) may also perform transmission/reception using a connector for wired communication. The sensing data and the basic index (SENSD) transmitted/received by the transmission/reception module (TRSR) are forwarded to and from the base station (GW) via the personal area network (PAN).

A display control (DISP) displays, on a display device (LCDD), the value of the basic index (TRIF) within the storage module (STRG). The displayed contents may also be switched by pressing the buttons (BTN1 to 3).

FIG. 3 is a diagram showing the block configuration of the sensor net server (14: FIG. 1) and the base station (20: FIG. 1). The base station 20 mediates the terminal and the sensor net server 14. When the terminal and the base station are connected wirelessly, a plurality of base stations are installed to cover areas such as the living space and the workplace in consideration of the wireless range. In the case of wired connection, the upper cap of the number of terminals to be managed is set according to the processing capacity of the base stations.

The base station 20 comprises a transmission/reception module (GWSR), a storage module (GWME) and a control response processing module (GWCO). The transmission/reception module (GWSR) receives data from the terminal via wireless or wired communication, and transmits the data to the sensor net server 14 via wired or wireless communication. When transmission/reception is performed wirelessly, the transmission/reception module (GWSR) will comprise an antenna for wireless reception.

The transmission/reception module (GWSR) performs congestion control, or timing control of communication, as needed to prevent the loss of data upon the transmission/reception of sensing data. The transmission/reception module (GWSR) classifies the type of received data. Specifically, the transmission/reception module (GWSR) identifies whether the received data is general sensing data, data for association, or a response of time synchronization from the header part of the data, and delivers each of the data to the appropriate function.

The storage module (GWME) is an external recording device such as a hard disk, a memory, or an SD card. The storage module (GWME) stores an operation setting (GWMA), data format information (GWMF), a terminal management table (GWTT), base station information (GWMG) and terminal firmware (GWTFD). The operation setting (GWMA) includes information representing the operating method of the base station 20. The data format information (GWMF) includes information representing the data format for communication, and information required for tagging the sensing data. The terminal management table (GWTT) includes terminal information (TRMT) of the terminal under control which is currently associated, and a local ID which is distributed for managing these terminals. When it is not necessary to connect with the terminal via wired connection and constantly comprehend the terminal (TR) under control, the terminal management table (GWTT) is not required.

The base station information (GWMG) includes information such as the address of the base station 20. The terminal firmware (GWTFD) stores a program for operating the terminal, and transmits (GWCFW) the firmware update data (TRDFW) to the terminal via the personal area network (PAN) upon receiving a command and new terminal firmware from the sensor net server 14.

The storage module (GWME) may also store programs to be executed by the CPU of the control module (GWCO). The control module (GWCO) comprises a CPU. The CPU executes the programs stored in the storage module (GWME) and manages the timing of receiving the sensing data from the terminal (TR), the processing of sensing data, the timing of transmission/reception to the terminal and the sensor net server 14, and the timing of time synchronization. Specifically, the CPU executes the processing of data reception control (GWCSR), data transmission (GWCSS), associate (GWCTA), terminal management information correction (GWCTF), terminal firmware update (GWCFW) and time synchronization (GWCS).

The timekeeper (GWCK) retains time information. The time information is updated in regular intervals. Specifically, the time information of the timekeeper (GWCK) is corrected based on the time information acquired from the NTP (Network Time Protocol) server (TS) in regular intervals.

The time synchronization (GWCS) transmits time information to the terminal under control in regular intervals, or when triggered by the terminal being connected to the base station 20. The time of the plurality of terminals and the time of the timekeeper (GWCK) of the base station 20 are thereby synchronized.

The associate (GWCTA) performs an associate response (TRTAR) of transmitting the assigned local ID to each terminal in response to an associate request (TRTAQ) sent from the terminal. Once the association is concluded, the associate (GWTA) performs a terminal management information correction (GWCTF) of correcting the terminal management table (GWTT).

The data reception control (GWCSR) receives a packet of the sensing data (SENSD) sent from the terminal. The data reception control (GWCSR) reads the header of the data packet, determines the type of data, and performs congestion control so that data from multiple terminals are not concentrated simultaneously.

A data transmission (GWCSS) assigns an ID and time data of the base station through which the data passed, and transmits the sensing data to the sensor net server 14.

The sensor net server 14 comprises a transmission/reception module (SSSR), a storage module (SSME), and a control module (SSCO). The sensor net server 14 manages data from all terminals. Specifically, the sensor net server 14 stores (SSCDB) the sensing data sent from the base station 20 in the sensing database (SSDB) based on a predetermined format (SSMF) (SSCDB). Furthermore, the sensor net server 14 searches for data in the sensing database (SSDB) based on a request from the application server (12: FIG. 1), and transmits the searched data to the application server 12 (SSDG).

Furthermore, the sensor net server 14 manages, as needed, information of the base station 20 and the terminal under its control (SSCTF), and becomes the source of the control command for updating the firmware of the terminal (SSCFW).

The transmission/reception module (SSSR) performs the communication control of data transmission/reception between the base station 20, the application server 12, and the client computer (16: FIG. 1).

The storage module (SSME) is configured from a data storage device such as a hard disk, and in the least stores the sensing database (SSDB), the data format information (SSMF), the terminal management table (SSTT) and the terminal firmware (SSFW). Furthermore, the storage module (SSME) stores programs to be executed by the CPU of the control module (SSCO).

The sensing database (SSDB) stores the sensing data acquired by each terminal, information of the terminal, and information of the base station 20 through which the sending data transmitted from each terminal has passed. The sensing database (SSDB) manages data based on a column for each data element such as acceleration, proximity information, and temperature. A table may also be used for each data element. In the database, the sensing data is managed by being associated with the terminal information (TRMT) and the detection time.

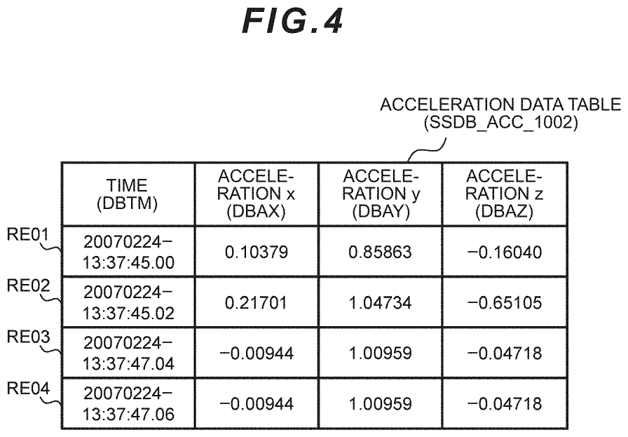

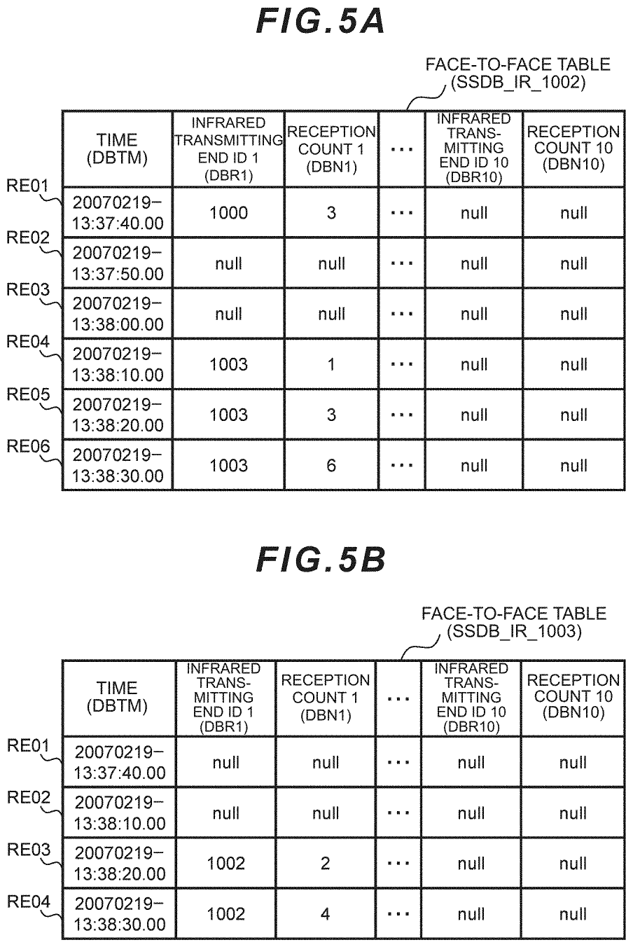

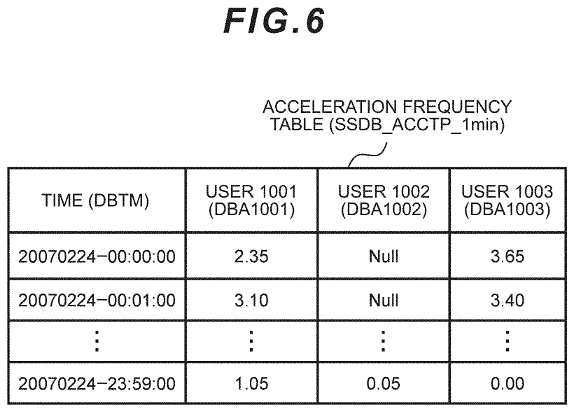

An example of the acceleration data table (table for each user) retained by the sensing database (SSDB) is shown in FIG. 4 (SSDB_ACC_1002: ACC represents the acceleration data, and 1002 represents the ID of the user (terminal TR)), an example of two people's worth of the infrared data table is shown in FIG. 5A (SSDB_IR_1002: IR represents infrared data, and 1002 represents the user ID) and FIG. 5B (SSDB_IR_1003), and an example of the acceleration frequency (or action rhythm) table for each person calculated from the acceleration data is shown in FIG. 6 (SSDB_ACCTP_1min).

The data format information (SSMF) stores information indicating the method of separately recording, in a database, the data format for the communication and the sensing data tagged by the base station 20, and the method of responding to the data request. The control module (SSCO) refers to the data format information (SSMF) after data reception and before data transmission, and performs data format conversion and data sorting.

The terminal management table (SSTT) stores information on which terminal is currently under the control of which base station 20. When a new terminal is added to be under the control of the base station 20, the control module (SSCO) updates the terminal management table (SSTT). Moreover, when the base station (GW) and the terminal (TR) are connected via wired connection, the control module (SSCO) is not required to monitor the terminal management information when the base station (GW) and the terminal (TR) are not connected.

The terminal firmware (SSFW) stores a program for operating the terminal. The terminal firmware update (SSCFW) updates the terminal firmware, and the transmission module transmits the updated terminal firmware to the base station 20 through the network 10. The transmission/reception module of the base station transmits the updated terminal firmware to the terminal through the personal area network (PAN). The terminal updates the firmware (FIG. 2: FMUD).

The control module (SSCO) comprises a CPU, and controls the recording and extraction of the sensing data to and from the transmission/reception module and the database. Specifically, as a result of the CPU executing the programs stored in the storage module (SSME), various types of processing such as data storage (SSCDB), terminal management information correction (SSCTF), terminal firmware update (SSCFW) and data acquisition/transmission (SSDG) are executed.

The data storage (SSCDB) receives the sensing data sent from the base station 20, and stores the received sensing data in the sensing database (SSDB). The data storage (SSCDB) stores as a single record, in the database, by combining additional information such as time information, terminal ID, and time of passing through the base station.

The timekeeper (SSCK) retains the standard time by periodically connecting to the external NTP server (TS). The terminal firmware update (SSCFW) and the data transmission (SSDG) may also be subject to timer activation (SSTK) when the designated time or specific condition is satisfied.

The terminal management information correction (SSCTF) updates the terminal management table (SSTT) upon receiving a command for correcting the terminal management information from the base station 20. The terminal management table (SSTT) comprises a list of terminals under the control of each base station 20.

The terminal firmware update (SSCFW) updates the terminal firmware (SSFW) in the storage module (SSME) manually or automatically when it becomes necessary to update the terminal firmware. Furthermore, the terminal firmware update (SSCFW) issues a command for updating the firmware of the terminal under the control of the base station 20. The terminal firmware update (SSCFW) receives a response from each terminal to the effect that the firmware update is complete. The terminal firmware update (SSCFW) continues the update until the update of all terminals is completed.

A configuration file (SSSF) stores information of the base station 20, and the terminal (TR) under its control, which is managed by the sensor net server (SS). When the configuration file (SSSF) is corrected, the configuration file (TRSF) in the terminal (TR) is updated using the channel of the terminal firmware update (SSCFW).

FIG. 4 described above shows an acceleration data table (SSDB_ACC_1002) as an example of the sensing data stored in the sensing database (SSDB) within the sensor net server 14. This table stores the actual sensing data acquired by the terminal. This table exists for each terminal (user), and stores the acceleration data in each of the triaxial directions of an X axis (DBAX), a Y axis (DBAY), and a Z axis (DBAZ) by associating such acceleration data with the time information (DBTM) for each sampling period (for example, 0.02 seconds).

Note that the table may also store the actual detected value of the acceleration sensor, or store the value after the unit has been converted into a gravitational constant [G]. This table stores the sensing time. The table may also be configured from a format which integrates a plurality of users based on a column indicating the user ID.

The sensing database (SSDB) stores multiple types of sensing data of each of a plurality of users. Among the above, an example of a table which summarizes the face-to-face data based on infrared transmission/reception is shown in FIG. 5A and FIG. 5B described above. FIG. 5A is a table indicating the collection of data acquired by the terminal having an ID of 1002, and FIG. 5B is a table indicating the collection of data acquired by the terminal having an ID of 1003. The terminal of ID 002 and the terminal of ID 003 are facing each other. If the column includes an infrared reception-side ID, the tables do not need to be separated for each terminal. Data related to the acceleration and temperature may also be included in the table.

The face-to-face tables of FIG. 5A and FIG. 5B each store the time (DBTM) that the terminal transmitted the data, the infrared transmission-side ID (DBR1) and the reception count (DBN1) of such ID (RE01, RE02 . . . ). The reception count represents the number of times infrared rays were received from which terminal every 10 seconds. Even when one terminal faced a plurality of terminals, the ID of each of the plurality of terminals is recorded in the table in one recording interval (10 seconds). When there is no face-to-face; that is, when a terminal did not receive infrared rays from another terminal, the terminal stores “null” in the reception count column.

FIG. 6 is an example of the acceleration frequency table (SSDB) storing the result of calculating the frequency for each predetermined time unit from the acceleration data table (SSDB_ACC) of FIG. 4 (SSDB_ACCTP_1min). The acceleration frequency table (SSDB_ACCTP_1min) stores, based on the acceleration data table (SSDB_ACC), the calculation result of the frequency for each fixed period (for instance, 1 minute) of each user (US) by associating the time per minute and the user ID. Note that the format for storing data may be, in addition to the foregoing table, a CSV file or other formats.

FIG. 7 is a diagram showing a hardware block configuration of the client computer 16, the application server 12, and the position detection sensor 18 (refer to FIG. 1 for these components).

The client computer 16 inputs and outputs data as the contact point with the management user. The client computer 16 comprises an I/O module (CLIO), a transmission/reception module (CLSR), a storage module (not shown), and a control module (CLCO).

The I/O module (CLIO) is an interface with the management user. The I/O module (CLIO) comprises a display (CLOD), a touch panel (CLIT), a keyboard (CLIK), and a mouse (CLIM). Another I/O device may be connected to an external I/O (CLIU) as needed.

The display (CLOD) is a CRT (Cathode-Ray Tube) or a liquid crystal display. The display (CLOD) may include a printer. The touch panel (CLIT) supports the input operation by the user. The touch panel (CLIT) may also be overlapped with the screen (OD: FIG. 1) of the display (CLOD) so that the output operation and the input operation can be performed on the same screen.

The transmission/reception module (CLSR) transmits/receives data and commands to and from the application server 12 and devices connected to another network. Specifically, the transmission/reception module (CLSR) transmits a request of the displayed screen to the application server 12, and receives an image corresponding to the request.

The storage module (not shown) is configured from an external recording device such as a hard disk, a memory, or an SD card. The storage module may also store the display history and the login ID of the management user.

The control module (CLCO) comprises a CPU, and performs processes such as control (CLCOD) of the screen to be output to the display (CLOD), and analyzing condition setting (CLCS) for the management user to notify the application server 12 of changing the analyzing condition.

The application server 12 performs calculation (ASGD) of the index of the group state, generation (ASCD) of the screen to be displayed on the client computer 16, and management (ASML) of the position detection sensor 18. The application server 12 comprises a transmission/reception module (ASSR), a storage module (ASME), and a control module (ASCO).

The transmission/reception module (ASSR) performs, through the network 10, communication control of data transmission/reception between the sensor net server 14, the NTP server (TS), the client 16, and the position detection sensor 18.

The storage module (ASME) is configured from an external recording device such as a hard disk, a memory, or an SD card. The storage module (ASME) stores the values of the calculation result, program for performing the calculation, and other data related to screen generation. Specifically, the storage module (ASME) stores position detection sensor information (ASLI), a display configuration file (ASDF), group state data (ASGS), a user attribute list (ASUL), area determination data (ASAD), and proximity determination data (ASND).

The position detection sensor information (ASLI) stores the ID, installed area and operational status of the position detection sensor 18 under control.

The display configuration file (ASDF) stores the image parts to be used in the screen design and configurations such as the display position in the display screen generation (ASCD).

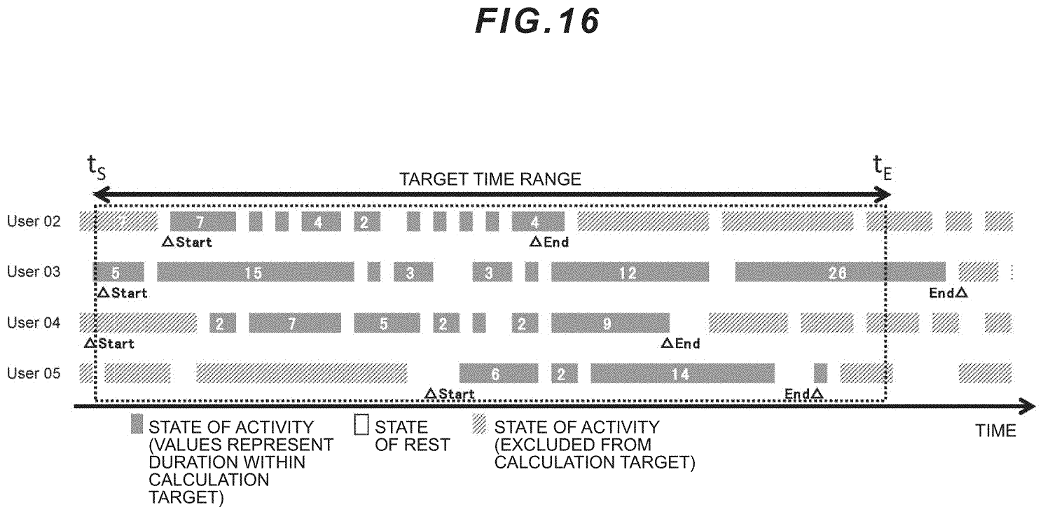

The group state data (ASGS) stores the group state index of a group in a specific area or related to specific persons. An example of the group state data related to people staying in a specific area is shown in FIG. 16 (ASGS_1). One or more position detection sensors (LS) are installed in an area, and associated based on the position detection sensor information (ASLI). The data to be calculated is determined based on the definition of the area stayers and the time range in the area determination data (ASAD).

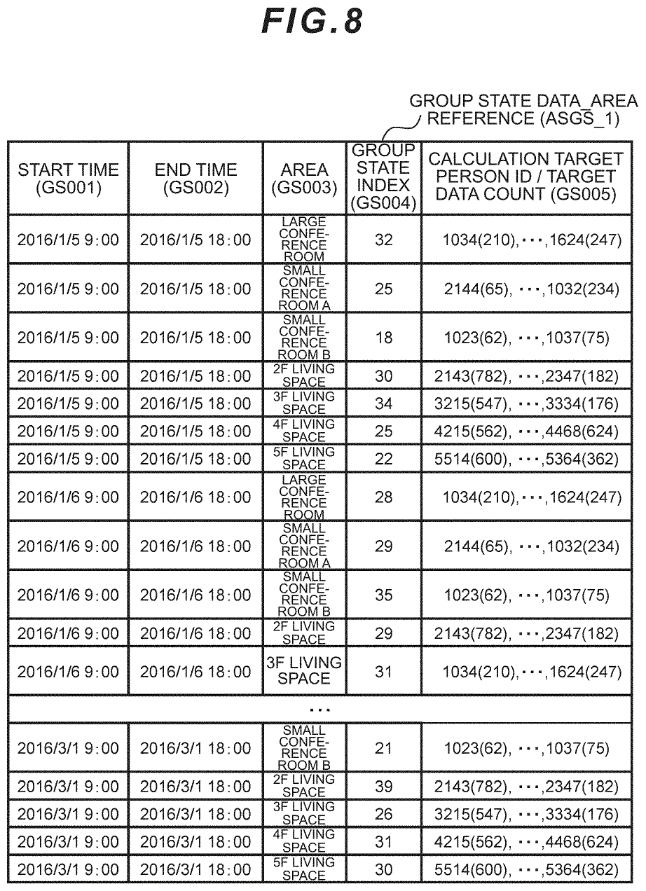

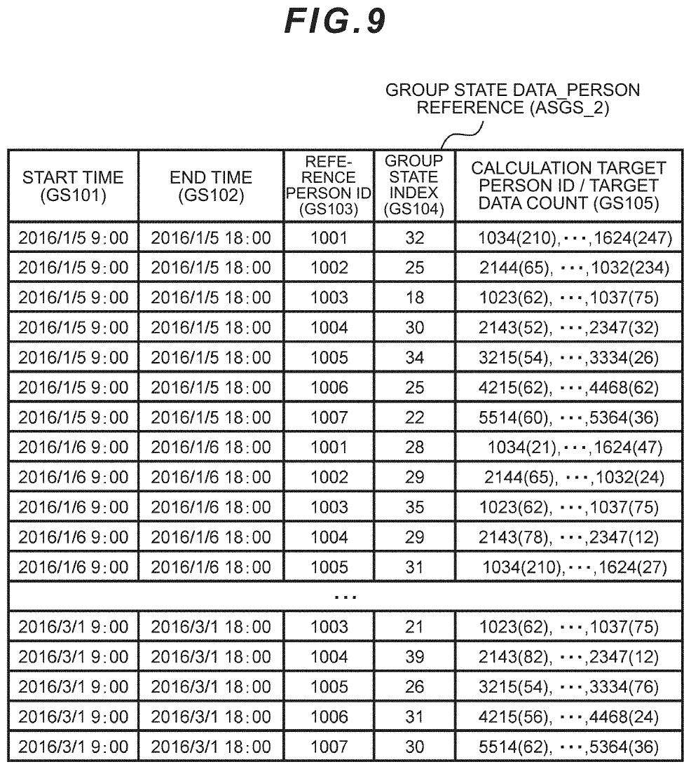

The group state data table (FIG. 8: ASGS_1) stores the start time (GS001) and the end time (GS002) of calculation of the group state index, area name (GS003), calculated value (GS004) of the group state index, and user ID and data count (GS005) of the data to be calculated. When the group state data indicates the group state of the people around a specific person, the format will be as per the group state data_person source table (ASGS_2) shown in FIG. 9. The main difference in comparison to the group state data_area source table (ASGS_1) is that the person ID (GS103) is indicated in substitute for the area ID. Furthermore, the proximity determination data (ASND) stores information (GS105) related to a person indicated as having approached that person during the period from the start time (GS101) to the end time (GS102), and the time of such approach.

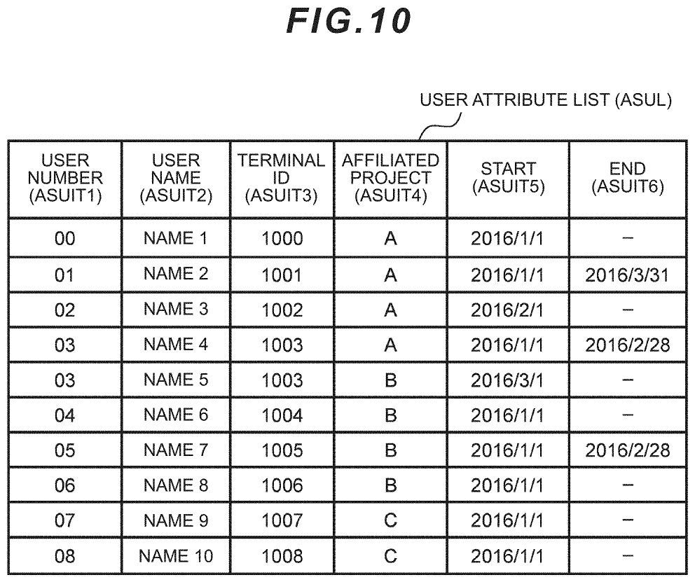

The user attribute list (ASUL) includes a comparative table of the terminal ID, and the name, user ID, affiliation, email address, and attribute of the user wearing that terminal. The user attribute list (ASUL) is referenced for associating the ID received from the counterparty upon facing such counterparty and the name, searching for the person affiliated with a predetermined business division, and the user logging onto the Web via the client computer. FIG. 10 shows a specific example thereof.

The specific example associates the user name (ASUIT2) with the user number (ASUIT1) and the held terminal ID (ASUIT3), and includes information of the affiliated project (ASUIT4) and the start (ASUIT5) and end (ASUIT6) of the period thereof. When the affiliated project (ASUIT4) is changed, the new project (ASUIT4) and period (ASUIT5, ASUIT6) are indicated by being associated with the same user number (ASUIT1).

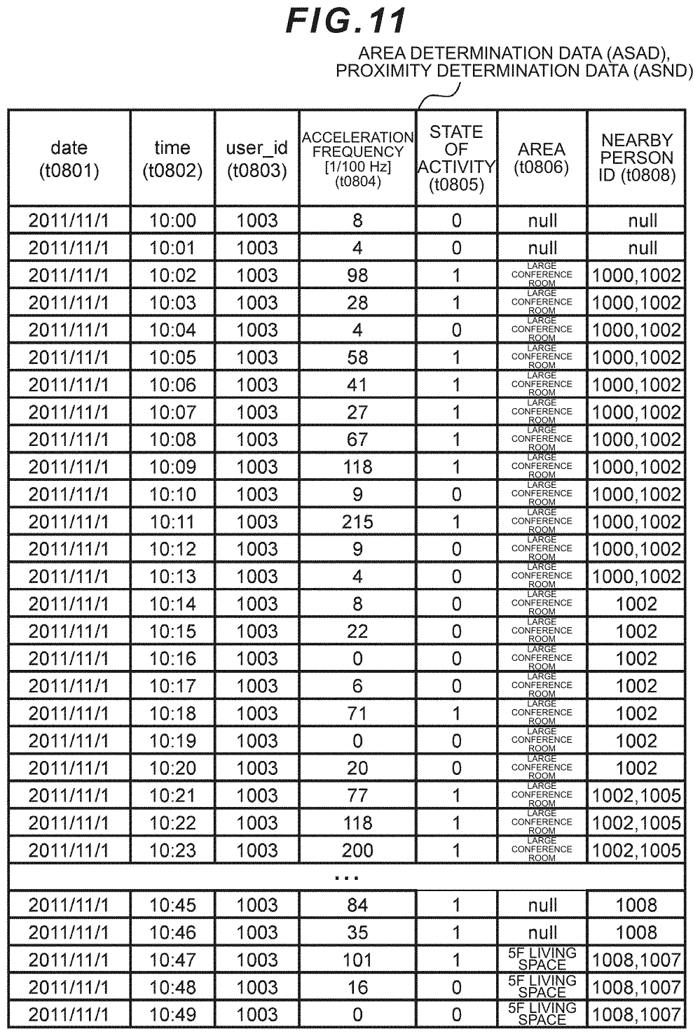

The area determination data (ASAD) shows the time and the area where the user has stayed. The proximity determination data (ASND) similarly shows the person who approached the user at such time. These may be separate files, or an integrated file. A format example of an integrated file is shown in FIG. 11. The area determination data may include the acceleration frequency (t0804) of the user at each point in time among the sensor data and the state of activity determination result (t0805) thereof acquired from the sensor net server 14.

The control module (ASCO) comprises a CPU, and executes processes such as data calculation, screen generation, and position detection sensor management. The application server 12 includes a timekeeper (ASCK), and maintains an accurate time by connecting to an external NTP server (TS) or the like. The control module (ASCO) performs timer activation (ASTK) and executes the program in the control module (ASCO) when it becomes the time that was present for each program. The activation method of the program may be manual, or be triggered based on a command from the client 16, or be triggered based on the data transmitted from the sensor net server 14 being a specific pattern.

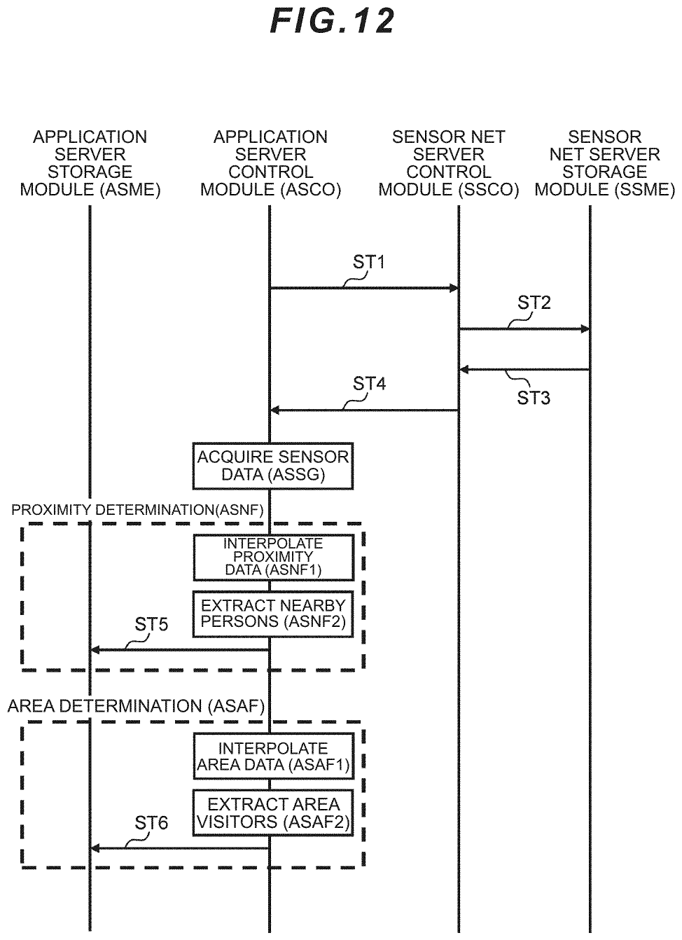

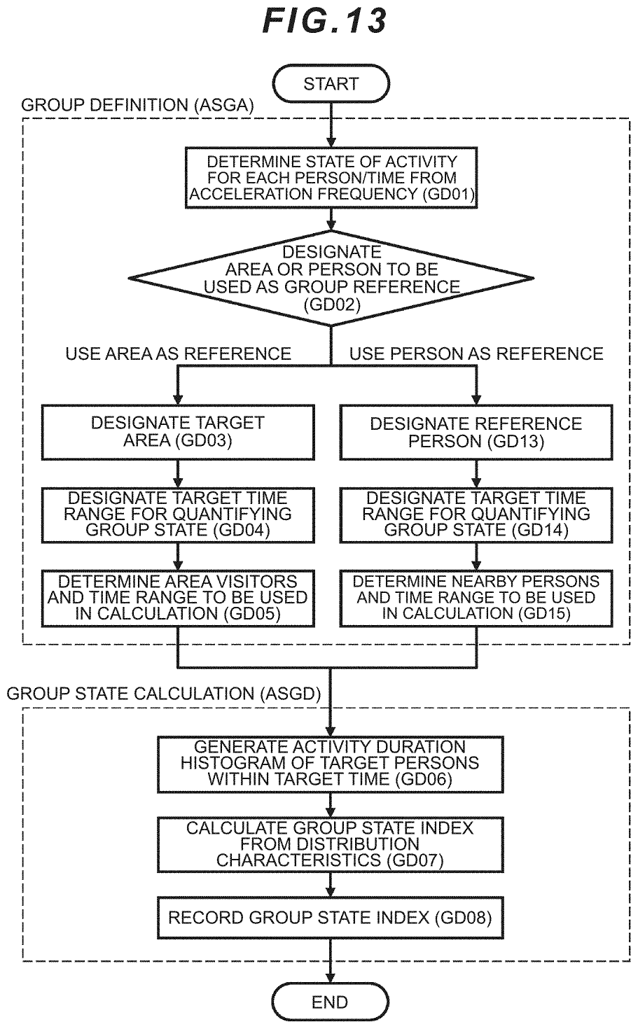

The process of calculating the group state index includes acquiring the sensor data (ASSG), performing proximity determination (ASNF) and/or area determination (ASAF), defining the group to be calculated (ASGA), calculating the group state (ASGD), and storing the calculation result in the group state data (ASGS). Details will be explained later with reference to the sequence diagram of FIG. 12 and the flowchart of FIG. 13.

The control module (ASCO), in the display screen generation (ASCD), associates the acquired sensor data with other business data acquired externally as needed, generates the group state index of the group state data (ASGS) in the form of a graph or the like, and displays the result on the screen data. Here, the display configuration file (ASDF) is referenced and the generated screen is transmitted to the client 16, and the client displays the generated screen (CLCOD) on the display (CLOD).

The position detection sensor management (ASML) manages the ID, installed area and operational status of the position detection sensor 18 under control, and stores these in the position detection sensor information (ASLI). Moreover, an operation/stop command may be sent to the position detection sensor 18. The position detection sensor management (ASML) may belong to the sensor net server 14 or an independent external server rather than the application server 12, and may be omitted if the position detection information is to be managed by the terminal.

The position detection sensor 18 is a device for identifying the user staying in a predetermined area, and includes a transmission/reception module (LSSR), a control module (LSCO), a storage module (not shown), and a wireless transmission/reception device (not shown).

The control module (LSCO) communicates with a terminal based on an infrared or wireless transmission/reception device (not shown) (LSWS), determines that the user (terminal owner) is staying in a predetermined area when the communication is established, stores data, by linking it to time information, in the storage module (not shown), and transmits such information to the application server 12.

The position detection sensor 18 may also identify a user staying in an area based on an image taken with a camera and a facial recognition program without comprising a wireless function. Moreover, the position detection information may also be received by the terminal. In the foregoing case, information of associating the user and the staying area and time may be transmitted from the terminal to the sensor net server 14, and the position detection sensor 18 may be separated from the network 10.

In order to calculate the index of the state of activity of the group in which a plurality of persons are related or involved based on the measured data of the plurality of persons, the information processing system defines, selects, determines or sets the group, and, for instance, determines a group among a plurality of options, subsequently prescribes the scope of calculation such as the scope of plurality of persons and the scope of time for calculating the index, extracts the measured data (for example, measured data of the wearable sensor) included in that scope, and thereafter calculates the index of the state of activity of the group based on the measured data. The information processing system determines the superiority or inferiority of the state of activity of the group; that is, whether the group is in an active state or a stagnant state, based on the size of the index value, or may entrust such determination to the user. The information processing system is advantageous over conventional systems as a result of being able to flexibly provide a plurality of references for determining the group. As the references for determining the group, for example, there are specific areas (conference room, venue, etc.), and specific persons. The information processing system defines the plurality of persons located in a specific area as a group in the case of the former, and defines the plurality of persons who approached the specific person as a group in the case of the latter. The information processing system can manifest and evaluate “flexible groups” such as a project team, gathering of volunteers, or teamwork of motivated persons which were previously unclear in comparison to office organization groups such as business divisions or sections.

FIG. 12 is a sequence diagram showing the data processing from the sensor data acquisition (ASSG) to the area determination (ASAF) by the application server 12. The application server control module (ASCO) starts the sequence based on timer activation (ASTK), or an activation command from the administrator, and transmits a sensor data request to the sensor net server 14 upon designating parameters such as the business division (scope of persons), period, data type, and temporal granularity to be calculated (ST1).

Based on the foregoing request, the sensor net server control module (SSCO) accesses the sensor net server storage module (SSME) (ST2), and acquires the sensing data corresponding to the request from the sensing database (SSDB) (ST3). The sensor net server control module (SSCO) transmits the sensing data to the application server control module (ASCO) (ST4).

In proximity determination (ASNF), the application server control module (ASCO) uses data related to the proximity of users; for instance, infrared face-to-face data or close-range wireless data between terminals, and extracts other users who could be deemed as having approached the specific user during a predetermined time range; for instance, on a daily basis (ASNF2). Because the proximity data may not be continuous depending on which direction the user's body is facing, the application server control module (ASCO) may also complement the lacking sections by performing smoothing thereto (ASNF1).

In nearby person extraction (ASNF2), the application server may store, in the recording module (ASME), the nearby person and the time of approach based on association as shown in FIG. 11, or store only the top three names who were nearby for the longest time during that day. Moreover, rather than indicating the proximity between two persons at each point in time as a binary value such as in the form of a column (t0808), the degree of proximity may also be indicated in a stepwise manner. The application server control module (ASCO) stores the thus extracted proximity determination data in the application server storage module (ASME) as the proximity determination data (ASND) by associating the ID of the nearby person with the time information or date information (ST5).

In area determination (ASAF), the application server control module (ASCO) similarly uses data related to the user's staying area acquired by the position detection sensor 18 and extracts the user who stayed in a specific area during a predetermined time range; for instance, on a daily basis (ASAF2). Here, because the area staying data may not be continuous depending on which direction the user's body is facing, the lacking sections may be complemented by performed smoothing thereto (ASAF1). In area stayer extraction (ASAF2), the user (US) who stayed in a specific area and the length of stay may be associated as shown in FIG. 11 and stored, or all persons who stayed for a predetermined time or longer during that day. The thus extracted area determination data is stored in the application server storage module (ASME) as the area determination data (ASAD) by associating the ID of the stayer with the time information or date information (ST6).

FIG. 13 shows a flowchart of group definition (ASGA) and group state calculation (ASGD). The application server control module (ASCO) compares the acceleration frequency detected by the acceleration sensor with a predetermined threshold for each individual and time unit after the start of the flowchart, determines that the individual is in a state of activity when the acceleration frequency is equal to or greater than the threshold, and determines that the individual is in a state of rest (state of inactivity) when the acceleration frequency is less than the threshold (GD01).

As an example, the routine of deriving (t0805) from the column (t0804) of FIG. 11 is shown. The threshold to be set here does not need to be uniform for all persons, and may be set individually such as by setting the daily acceleration frequency of each individual as the threshold, or may be defined depending on the job title. Moreover, while the time unit is 1 minute in FIG. 11, the time unit is not limited thereto. “1” of the state of activity (t0805) in FIG. 11 represents a state of activity and “0” represents a state of inactivity. The duration in which the acceleration frequency is equal to or greater than the threshold is the activity duration.

Note that an acceleration sensor is merely an example of a sensor for detecting the state of activity of an individual, and a microphone which detects the loudness of statements made by an individual may otherwise be used as the sensor.

Next, the application server control module (ASCO) selects whether the reference for defining the group will be “area” or “person” (GD02). This classification is merely an example, and is not limited thereto. This classification may also be made by the management user.

When the application server control module (ASCO) selects “area” as the source, it designates the target area (GD03), and designates the target time range upon quantifying the group state (GD04). The target time range is, for example, from 0:00 to 24:00 of the same day when calculating one group state index in 1-day units.

The application server control module (ASCO) designates the area target person and time range for calculating the index (GD05). The area target person may be a person who may enter the specific area. For example, this would be a person affiliated with a business division or section.

As a result of the application server control module (ASCO) referring to the area determination data (ASAD) and extracting a user who has been recorded as staying in a predetermined area within the target time range and extracting the start time and end time that such user stayed in that area, the state of activity data of the user during his/her stay in the area can be included in the scope of calculation.

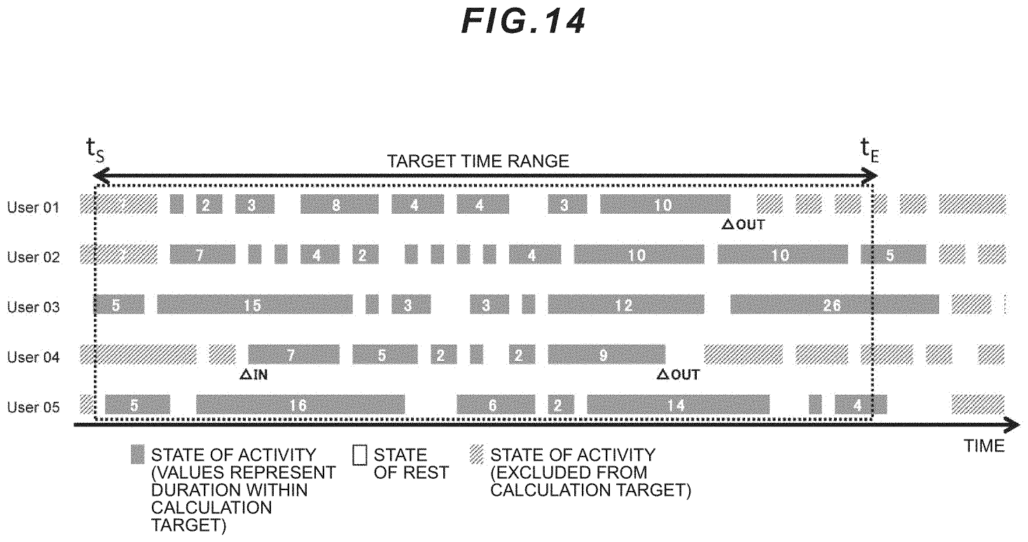

One mode for determining the scope of calculation in step (GD05) is now explained with reference to FIG. 14. FIG. 14 shows whether each of the plurality of users (User 01 to User 05) who were recorded as staying in the area in the target time range (t_{S} to t_{E}) was in a state of activity or a state of rest in time units, and the duration (seconds) of the state of activity. In FIG. 14, IN represents the timing of entering the area, and OUT represents the timing of exiting the area. If there is no indication of IN, OUT during the target time range (t_{S} to t_{E}), it means that a user had already entered the area and had not existed the area during the target time range (t_{S} to t_{E}). As a general rule, the “duration” between t_{S} or IN, whichever is earlier, and between t_{E} or OUT, whichever is later, is used for calculating the index.

In a mode where the time from IN to OUT is included in t_{S} to t_{E} as with User 04, the data from IN to OUT will be the scope of calculation of the state of activity of the group, and the application server control module (ASCO) sets the calculation start time of User 04 to the timing of IN, and sets the calculation end time to the timing of OUT.

In a mode where the time of IN is before t_{S} and the time of OUT is between t_{S} and t_{E} as with User 01, the calculation end time will be the time of OUT on the one hand, and, when the state of activity has been ongoing from the past at the time of t_{S}, the timing of starting the next state of activity after the completion of the ongoing state of activity or the timing that the previous ongoing state of activity has ended is defined as the calculation start time. The foregoing configuration is adopted because the state of activity before t_{S} is unrelated, or unlikely to be related, to the activity of t_{S} onward. However, this configuration is not mandatory, and t_{S} may be used as the timing for starting the calculation.

In a mode where the user has not exited the area and the state of activity is ongoing at time t_{E} as with Users 02, 03, 05, the time in which the ongoing state of activity is completed (time later than t_{E}) will be the timing of ending the calculation. This is in order to prevent the duration count being discontinued midway when the duration count is uniformly stopped at t_{E} in subsequent routines (GD06), (GD07) of FIG. 13 despite the fact that, in effect, the individual's ongoing state of activity is continuing. In the histogram (example of a statistical distribution profile) of the activity duration generated in subsequent routine (GD06), because the frequency that a long activity duration is generated is low, the influence on the distribution profile will be considerable even when the foregoing discontinuation occurs even once, and the numerical value of the group state index will fluctuate significantly.

Note that, in routine (GD05), the application server control module (ASCO) may include the entire duration of t_{S} to t_{E} in the scope of calculation so as long as a user stayed in the area during the target time range (tₛ to t_{E}) for a predetermined time or longer, irrespective of whether that user entered the area after t_{S} and irrespective of whether that user exited the area before t_{E}.

Subsequent to routine (GD05), the application server control module (ASCO) generates a histogram of the area stayer by totaling the activity duration count from the calculation start time to the calculation end time generation (GD06), calculates the distribution characteristics of the histogram as the group state index (GD07), stores the index in the group state data (ASGS) (GD08), and then ends the series of processing.

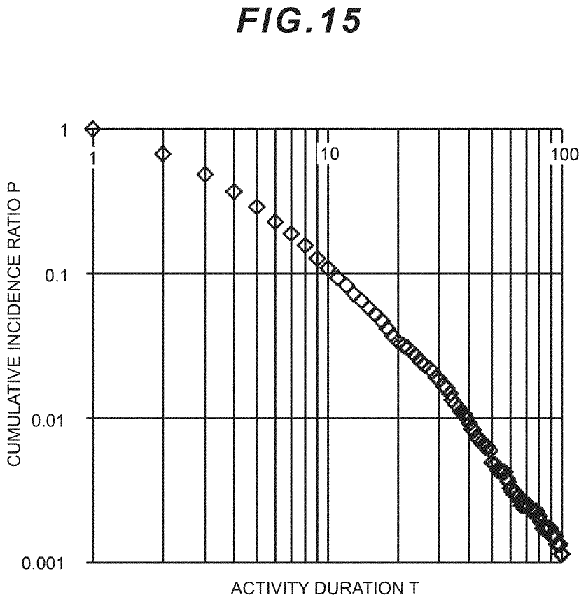

An example of the form of the histogram generated in routine (GD06) is shown in FIG. 15 (area: large conference room; period: 5/12 9:00-1700). The application server control module (ASCO) calculates a cumulative value by totaling all activity durations within the calculation target period for each of the plurality of user to be subject to the calculation of the group state index, and creates a histogram of the activity duration T and the cumulative incidence ratio P. FIG. 15 is a graph in which the activity duration T is indicated as the horizontal axis and the cumulative incidence ratio P is indicated as the vertical axis, and indicates the two as a log. The application server control module (ASCO) may also perform weighting to the activity duration according to the average value of the acceleration frequency of individuals, or superimpose the histogram after normalizing the activity duration.

In routine (GD07), the application server control module (ASCO) calculates the amount of characteristic related to the distribution profile, for example, one or more values related to the incidence ratio, inclination or curvature of a specific section, or cutoff position when the activity duration T is of a specific value or in a specific scope, and calculates the value of the group state index based on a predetermined function with the foregoing values as variables. The predetermined functions may be defined in advance so that they coincide with the values of the questionnaire results related to the individual's stress or productivity acquired separately from the user (US) by multiplying the foregoing amount of characteristic by a coefficient. The calculation of the index value based on the amount of characteristic of the distribution profile of the histogram may also be performed according to the methods disclosed in PTL 1, 2 described above. Obtaining the group index based on a histogram is merely an example, and is not limited thereto.

As an example of the above, it is possible to define the histogram of an invigorated group, and deem that a group is not invigorated when the incidence ratio of T having a long activity duration is low or the incidence ratio of T having a short activity duration is high in comparison to the histogram of the invigorated group. As a specific example, it is highly likely that a group including many persons whose length of concentration is extremely short and intermittent is not invigorated.

In routine (GD02), when the application server control module (ASCO) selects a group in which a person is used as the reference, it is possible to calculate an index indicating the dynamic state of the group of approaching the reference person of the group resulting from the person's characteristics such as personality and role. In the foregoing case, in the same manner as routines (GD03 to GD05), the application server control module (ASCO) selects the person to be used as the reference (GD13), designates the target time range (t_{S} to t_{E}) in the same manner as GDO4 (GD14), and thereafter determines the person who approached the reference person within the target time range (t_{S} to t_{E}) and the time range to be used for the calculation based on the proximity determination data (ASND) (GD15).

An example of the mode of determining the scope of calculation in routine (GD15) is now explained with reference to FIG. 16. FIG. 16 shows the reference person as User 01, the start time that each person started to approach User 01 as “Start”, and the end time that each person moved away from User 01 as “End”. While FIG. 14 used the stay in a specific area as the reference, based on the same method, the time range of being nearby User 01 is defined as the scope of calculation of each user (US). With regard to User 01, the entire target time range (t_{S} to t_{E}) is defined as the scope of calculation.

As shown in FIG. 16, the scope of calculation is determined for each user who approaches User 01. The duration of the state of activity including the state of activity belonging to “Start” to the state of activity belonging to “End” regarding User 02, User 03, and User 05 is the calculation target. With regard to User 03, while “End” has occurred after t_{E}, because the state of activity is ongoing at time t_{E}, the duration of this state of activity is also included in the calculation target. With regard to User 04, even if “Start” has occurred before t_{S} and the state of activity is ongoing at time t_{S}, the duration of this state of activity is not included in the calculation target.

In routine (GD15), rather than setting the calculation target time for each User, the duration of persons (User 02 to User 05) who approached the reference person (User 01) during the target time range (t_{S} to t_{E}) for a predetermined time or longer between t_{S} and t_{E} may also be used as the calculation target. The reason for this is because a person who was nearby User 01 for a predetermined time or longer is likely to have been relatively close to User 01, such as being on the same floor, even during the time period that proximity was not detected (outside the scope of “Start” to “End”).

The application server control module (ASCO) performs same routines (GDO6 to GD08) as those described above by using the data of the activity duration within the calculation target period corresponding to each target person defined in routine (GD15).

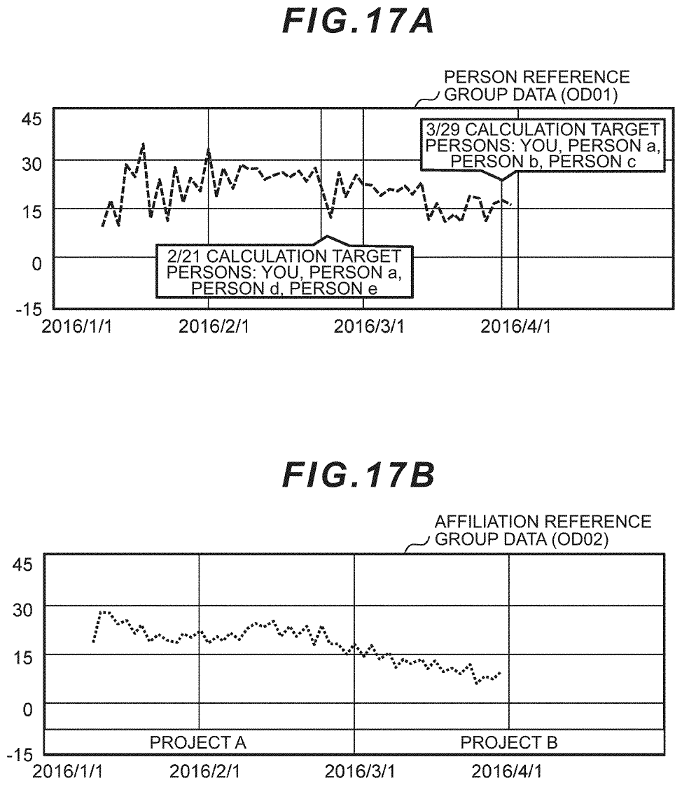

Next, an example of the display screen (OD) of the Web application generated by the foregoing display screen generation (ASCD) is shown. FIG. 17A, FIG. 17B, and FIG. 17C show a mode of visualizing the changes in the value of the group state index of a specific dynamic group with a polygonal line graph. The person reference group data (OD01: index of dynamic group) of FIG. 17A shows data resulting from selecting a person as the reference in routine (GD02) of FIG. 13, the affiliation reference group data (OD02: index of fixed group) of FIG. 17B shows data resulting from a group affiliated with the viewer (User 0203) according to the definition of the affiliated project (ASUIT4) of FIG. 10, and the area reference group data (OD03: index of dynamic group) of FIG. 17C shows data resulting from selecting an area as the reference in routine (GD02) of FIG. 13. In FIG. 17A, FIG. 17B, and FIG. 17C, the target time range is 1 day, and changes of individual data are shown.

In these data, the target person to be the subject of index calculation may differ depending on the day, and, in such as a case, the application server 12 may refer to the data table and extract the target persons and display such persons in a balloon as shown in the diagrams. Consequently, in the person reference group data (FIG. 17A: OD01) and the affiliated project (FIG. 17B: ASUIT4), there is an advantage in that the user (User 03) can intuitively and easily recognize, based on changes in the graph, the activity level with others centered around himself/herself, such as the level of energy or fatigue.

In the area reference group data (FIG. 17C: OD03), the application server 12 can refer to the data table and extract the target person, and superimpose and display the number of participants of the meeting and the agenda of the meeting on the graph. For example, by comparing the indexes for each area, the result can be utilized for the layout change of the area, such as the arrangement of desks and use of wallpaper.

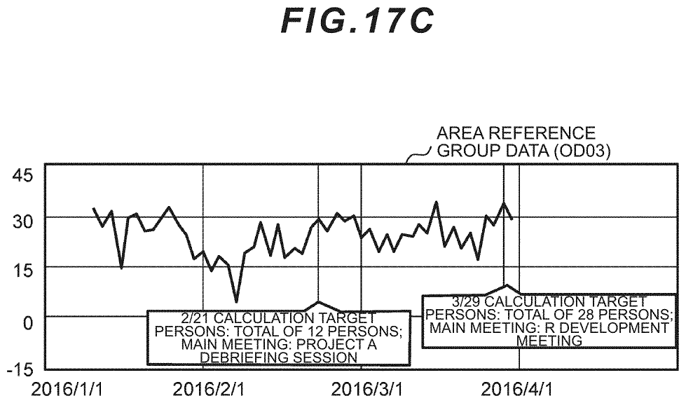

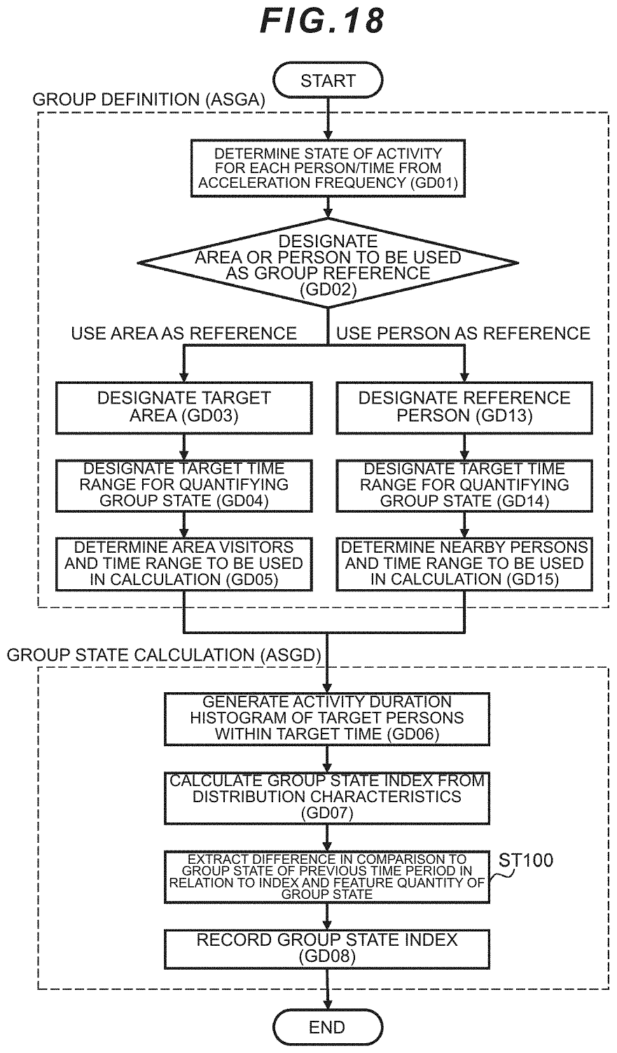

Another mode of FIG. 14 is now explained. FIG. 18 shows a flowchart of adding a step (ST100) between step (GD07) and step (GD08) of the group state calculation block (ASGD) of FIG. 14. This step (ST100) is the processing for displaying the difference of the indexes on the graph (FIG. 17) showing the changes in the group state index. A display example is shown in FIG. 19. This display example includes a text balloon (difference display) describing the difference on how much the group state index (integrated value) of the target period (for example, 2/21 to 2/24) has increased or decreased relative to the group state index (integrated value) of the comparative period (for example, 1 week before the target period, or same number of days in the previous month).

In order to realize this difference display, for example, the application server 12 calculates the difference of a plurality of target periods, and displays a balloon for the target period having a difference of a predetermined level or greater relative to each of the comparative periods. The target period and the comparative period may be determined by the application server 12, or selected by the management user.

The application server 12 calculates the cause of difference and also displays such cause on the graph. In FIG. 19, “Decrease of ∘% from last week” is the difference, and “It appears that person A's degree of involvement has decreased in comparison to last week” corresponds to the cause. The application server 12 identifies the cause based on the amount of characteristic (for example, group member, member's degree of involvement) that affects the difference. The “degree of involvement” is the level of influence that the sensor data (proximity information) of the group member has on the group state index.

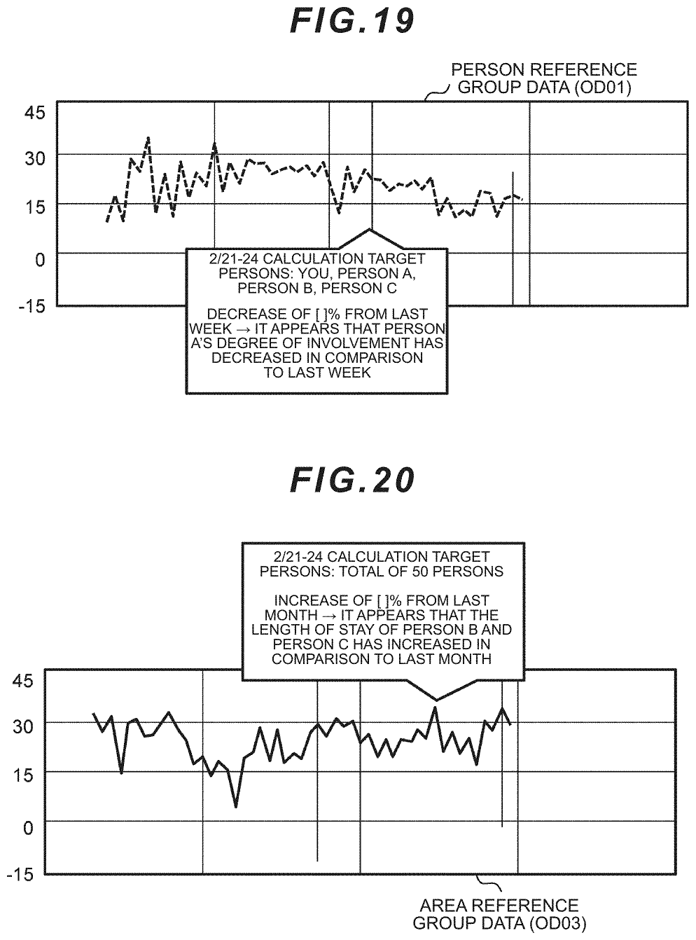

FIG. 20 is a graph showing changes in the index pertaining to another mode of the display example. While the foregoing display example (FIG. 19) is data of a group that uses a person as the reference, this display example relates to data of a group that uses a specific area as the reference. “2/21 to 2/24 Calculation target persons: Total of 50 persons” is an explanation of the group in the target period, “Increase of ∘% from last month” is the difference, and “Stay rate (involvement) of person B and person C has increased in comparison to last month” corresponds to the cause. The comparative period may be, for example, one or more among daily, weekly, monthly and annually.

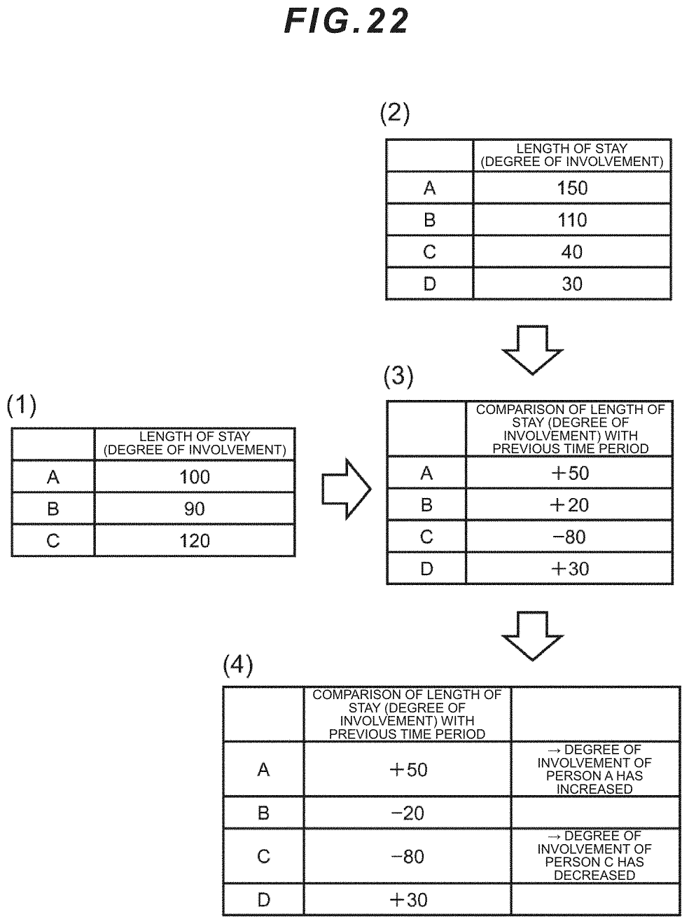

In the display example of FIG. 20, the application server control module (ASCO) may calculate the foregoing cause based on the length of stay in the area. The application server control module (ASCO) scans the database of the detected values of the position detection sensor 18, respectively identifies the length of stay in the area for each of persons A to C in the previous period (2/21 to 2/24) (degree of involvement): FIG. 22(1)), and the length of stay in the area for each of persons A to D (degree of involvement): FIG. 22(2)) in the current period (2/14 to 2/17), and calculates the difference between the two. FIG. 22(3) is a table summarizing the foregoing differences, and, for example, (A: +50) represents that person A's degree of involvement has increased by 50%, and a negative value represents that the person's degree of involvement has decreased. The application server control module (ASCO) compares the absolute value of the increase/decrease of the degree of involvement with the threshold, and superimposes and displays a corresponding message (refer to FIG. 22(4)) on the graph upon exceeding the threshold.

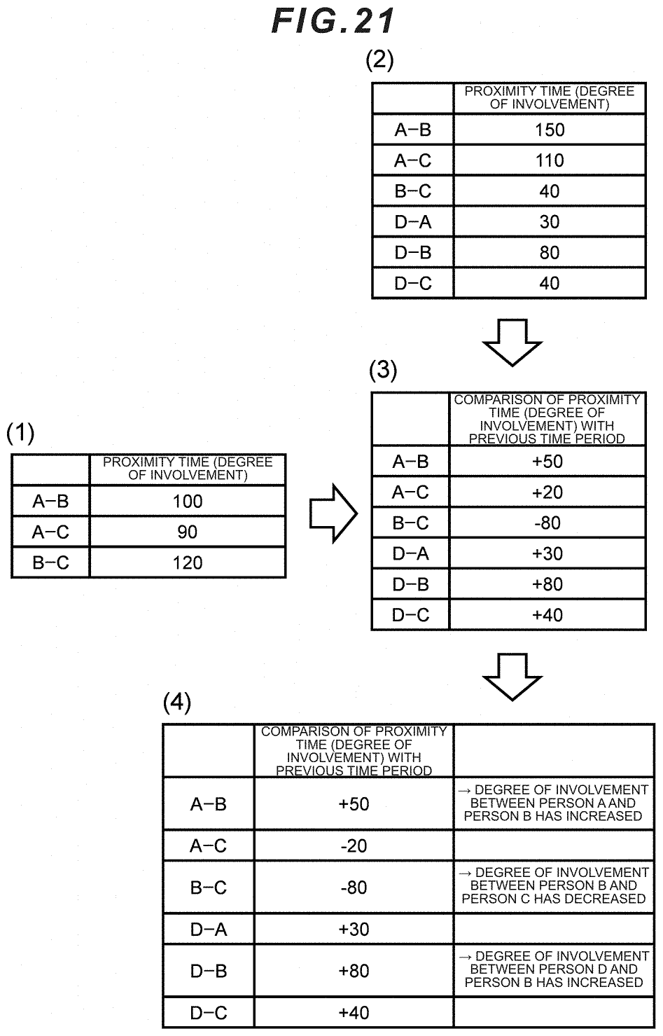

Another mode of the method for identifying the cause of difference in the group state index is now explained. In the foregoing mode, the application server control module (ASCO) analyzed the influence for each of the plurality of nearby persons around the central person in the mode of a group using a person as the reference, and analyzed the influence for each of the plurality of persons staying in the area in the mode of a group using an area as the reference. Meanwhile, in this other mode, the application server control module (ASCO) identifies the cause by comparing the persons around the central person, or comparing the proximity time (face-to-face time) of persons staying in the area with the previous period and the current period. In FIG. 21, (1) shows the degree of involvement between persons in the previous time period, and for instance “A-B” represents the proximity time (degree of involvement) between person A and person B. (2) shows the degree of involvement between persons in the current time period. The application server control module (ASCO) compares the degree of involvement of both time periods (FIG. 21(3)), compares the result with the threshold, and superimposes and displays a predetermined message (FIG. 21(4)) on the graph.

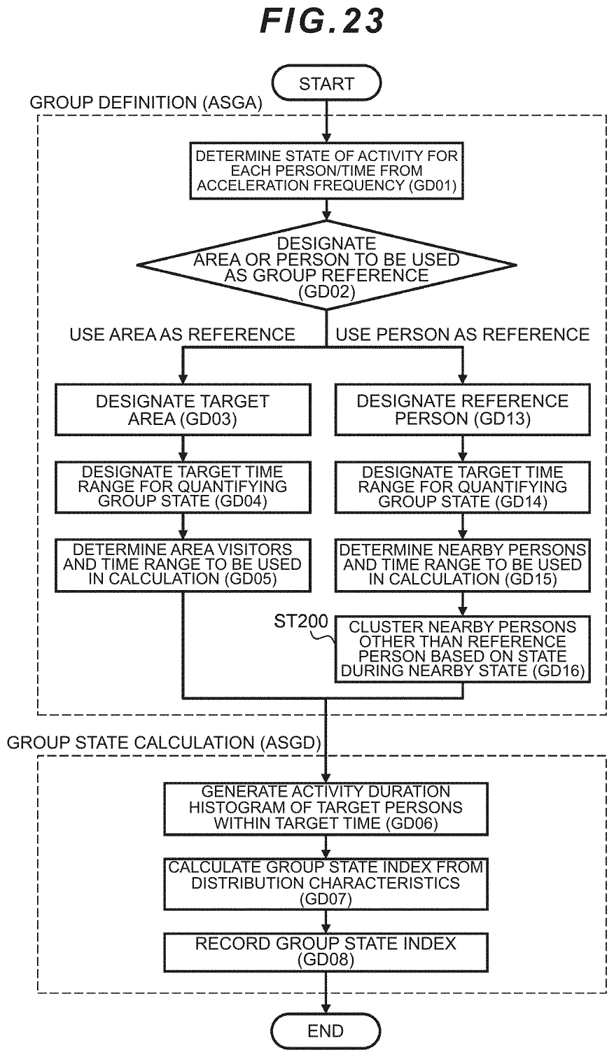

In a group using a person as the reference, the application server control module (ASCO) can manifest a dynamic group in relation to the group using a person as the reference by clustering the proximity or face-to-face of nearby persons relative to the reference person (FIG. 23, ST200).

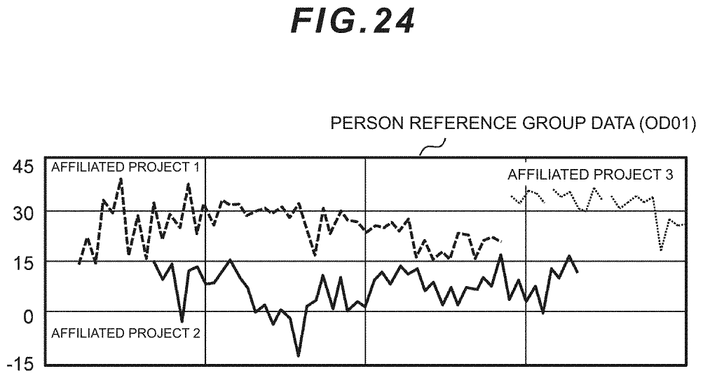

As case 1, assumed is a mode in which A and B, A and C, A and D, B and C, B and D, C and D are near each other (persons A to D are nearby persons of the same group). The application server control module (ASCO) determines that persons A to D are mutually near each other from the proximity data of nearby persons, and determines that all persons A to D, including the reference person, are affiliated with one affiliated project (one group: affiliated project 2 of FIG. 19, FIG. 21, and FIG. 24).

Next, as case 2, assumed is a mode where, while A and B, C and D are near each other, no other persons are nearby. The application server control module (ASCO) can know of the existence of a group of the reference person, A, and B (affiliated project 1) and a group of the reference person, C, and D (affiliated project 3) in addition to the group of the reference person and A to D (affiliated project 2). Note that the application server control module (ASCO) generates the graphs of the affiliated projects 1, 3 in the same manner as the graph of the affiliated project 2.

The embodiments of the present invention have been explained above, but the present invention is not limited to the foregoing embodiments, and it can be understood by those skilled in the art that the present invention can be variously modified, and that each of the foregoing embodiments may be combined as needed.

### REFERENCE SIGNS LIST

TR, TR2 to 3 terminal
GW base station
US, US2 to 3 user
NW network
PAN personal area network
SS sensor net server
AS application server
CL client

1. An information processing system, comprising:
a recording device which collects, and stores, data via a network from a terminal device worn by each of a plurality of persons; and
a computer which sets a group of a predetermined number of persons among the plurality of persons based on the collected data,
wherein the terminal device outputs measured data of the person to the network,
wherein the recording device stores the measured data, and
wherein the computer calculates an index of a state of activity of the group based on the measured data of the terminal device worn by each person belonging to the group.

2. The information processing system according to claim 1, further comprising:
a detection device which detects a terminal device in a predetermined area and outputs information of the detected terminal device to the network,
wherein the recording device stores information of the detected terminal device, and
wherein the computer sets persons possessing terminal devices in the predetermined area as the group based on information of the detected terminal device.

3. The information processing system according to claim 1,
wherein the computer selects a person to become a reference among the plurality of persons,
wherein, when a terminal device of the reference person detects that a terminal device of another person is in close proximity, the terminal device of the reference person outputs information of the terminal device of the other person to the network,
wherein the recording device stores information of the terminal device of the other person, and
wherein the computer sets the other person in the group of the reference person based on the information.

4. The information processing system according to claim 1,
wherein the terminal device comprises an acceleration sensor, and the acceleration sensor outputs, as the measured data, data of acceleration of motion of the person to the network,
wherein the recording device stores the acceleration data, and
wherein the computer calculates an index of a state of activity of the group based on the acceleration data of the terminal device worn by each person belonging to the group.

5. The information processing system according to claim 4,
wherein the computer determines a state of activity of each person belonging to the group based on the acceleration data, and calculates an index of a state of activity of the group according to the state of activity of each person belonging to the group.

6. The information processing system according to claim 5,
wherein the computer calculates an index of a state of activity of the group according to a duration of the state of activity of each person belonging to the group.

7. The information processing system according to claim 1,
wherein the computer sets a predetermined time range, and calculates an index of a state of activity of the group based on the measured data which was measured by the terminal device within the time range.

8. The information processing system according to claim 6,
wherein the computer sets a predetermined time range, and calculates an index of a state of activity of the group based on the measured data which was measured by the terminal device within the time range, and
wherein the computer, when a state of activity of at least one person belonging to the group is ongoing when the time range reaches a termination number, calculates an index of a state of activity of the group including a duration until the state of activity is ended.

9. The information processing system according to claim 3,
wherein the computer refers to information from terminal devices, by accessing the recording device, associated with proximity of persons other than the reference person of the group, and sets the group based on information from the terminal devices.

10. The information processing system according to claim 1,
wherein the computer refers to information from terminal devices, by accessing the recording device, associated with proximity of persons configuring the group, and evaluates the index based on the terminal devices.

11. The information processing system according to claim 1,
wherein the computer refers to information from terminal devices, by accessing the recording device, associated with proximity of persons configuring the group, and evaluates the index based on information from the terminal devices.

12. The information processing system according to claim 2,
wherein the computer refers to a length of stay in the area of the persons configuring the group, and evaluates the index based on information of the length of stay.

13. An information processing method using a computer system,
wherein the computer system:
collects, and stores, data via a network from a terminal device worn by each of a plurality of persons;
sets a group of a predetermined number of persons among the plurality of persons based on the collected data;
stores measured data of the person which was output from the terminal device to the network; and
calculates an index of a state of activity of the group based on the measured data of the terminal device worn by each person belonging to the group.
